(19) United States
(12) Patent Application Publication
(10) Pub. No.: US 2026/0216711 A1
(43) Pub. Date: Jul. 30, 2026

(54) FERRIERITE (FER)-TYPE ZEOLITES AND THE USES THEREOF

(71) Applicant: BRASKEM AMERICA, INC., Philadelphia, PA (US)

(72) Inventors: Mengyao OUYANG, Pittsburgh, PA (US); Priya D. SRINIVASAN, Pittsburgh, PA (US); Dan XIE, Pittsburgh, PA (US); Nailma DE JESUS MARTINS, Sao Paulo (BR); Gabriela MACIEL FUZINA, Sao Paulo (BR); Jiahan XIE, Pittsburgh, PA (US)

(21) Appl. No.: 19/461,227

(22) Filed: Jan. 27, 2026

Related U.S. Application Data
(60) Provisional application No. 63/749,913, filed on Jan. 27, 2025.

(51) Int. Cl. B01J 29/65; B01J 35/61; B01J 35/63; B01J 37/10; B01J 37/30; C01B 39/02; C01B 39/44; C07C 1/24; C07C 11/107; C07C 11/113

(52) U.S. Cl. CPC B01J 29/65; B01J 35/615; B01J 35/633; B01J 37/10; B01J 37/30; C01B 39/026; C01B 39/44; C07C 1/24; C07C 11/107; C07C 11/113; B01J 2229/12; B01J 2229/18; B01J 2229/38; C01P 2002/72; C01P 2006/12; C01P 2006/14; C07C 2529/65

(57) ABSTRACT

This disclosure relates to a modified ferrierite (FER)-type zeolite comprises micropores and mesopores, with a total mesopore volume greater than about 0.05 cm³/g. The zeolite is modified by a property-enhancement treatment involving mixing with hydroxide ions and a surfactant, followed by heating. This disclosure also relates to a fatty alcohol dehydration process comprising providing a feedstock containing a fatty alcohol and dehydrating the fatty alcohol in the presence of the modified FER-type zeolite catalyst to obtain a dehydration product.

### CROSS REFERENCE TO RELATED APPLICATIONS

This application claims benefit under 35 U.S.C. § 119 (e) to U.S. provisional patent application No. 63/749,913, filed on Jan. 27, 2025, herein incorporated by reference in its entirety.

### FIELD OF INVENTION

This disclosure generally relates to zeolite catalysts and the use thereof in catalytic processes.

### BACKGROUND OF THE INVENTION

Zeolites are typically microporous, crystalline aluminosilicate materials that have found widespread use as catalysts and adsorbents in various industrial processes. The catalytic application of zeolites depend highly on their size, shape, and surface area.

Fatty alcohols are common biobased intermediates in the chemical industry that may be derived from various natural sources such as plant oils, animal fats, sugar, and/or cellulose, serving as precursors for various products including thermoplastics, surfactants, lubricants, and plasticizers, etc. The dehydration of fatty alcohols to produce olefins is a valuable transformation, as olefins are versatile building blocks for numerous chemical processes. The olefins produced can be alpha-olefins (e.g. CH₂═CH(R), where R is hydrogen or a carbon-containing group), internal olefins (e.g. CH₃—CH═CH—CH₃), or branched olefins (e.g. CH₃—C(═CH₂) (—CH₃)). In particular, linear alpha olefins are promising fatty-alcohol-derived product due to its high market demand. To obtain linear alpha olefin (e.g., a C₃-C₁₈ alpha olefin, or a C₄-C₈ alpha olefin), fatty alcohol is typically first converted to linear olefins via dehydration, followed by other reactions such as oligomerization, isomerization, cracking, and cross-metathesis with ethylene.

Solid acid catalysts, including zeolites, have been explored for fatty alcohol dehydration reactions. However, achieving high efficiency production and high selectivity towards desired olefin products, while minimizing undesirable byproducts, remains a challenge in fatty alcohol dehydration processes.

There is an ongoing need in the field for an improved catalyst that can enhance the efficiency and selectivity of fatty alcohol dehydration processes.

### SUMMARY OF THE INVENTION

According to one aspect of the disclosure, a modified ferrierite (FER)-type zeolite is provided. The modified FER-type zeolite comprises a FER-type zeolite, modified by a property-enhancement treatment. The modified FER-type zeolite has micropores and mesopores (such as mesopores having an average pore-diameter of greater than 2 nm; e.g., an average pore-diameter ranging from 2 to 50 nm), wherein the total mesopore volume is greater than about 0.05 cm³/g (e.g., about 0.06 cm³/g or greater, about 0.10 cm³/g or greater, or about 0.14 cm³/g or greater), measured by Ar adsorption isotherms obtained at 87K, or by N₂ adsorption isotherms obtained at 77K, on a Surface Area and Porosity Analyzer.

According to another aspect of the disclosure, a process for a fatty alcohol dehydration is provided. The process comprises providing a feedstock containing a fatty alcohol, and dehydrating the fatty alcohol in the feedstock in the presence of a modified ferrierite (FER)-type zeolite catalyst to obtain a dehydration product. The modified FER-type zeolite comprises a FER-type zeolite, modified by a property-enhancement treatment. The modified FER-type zeolite has micropores and mesopores (such as mesopores having an average pore-diameter of greater than 2 nm; e.g., an average pore-diameter ranging from 2 to 50 nm), wherein the total mesopore volume is greater than about 0.05 cm³/g (e.g., about 0.06 cm³/g or greater, about 0.10 cm³/g or greater, or about 0.14 cm³/g or greater), measured by Ar adsorption isotherms obtained at 87K, or by N₂ adsorption isotherms obtained at 77K, on a Surface Area and Porosity Analyzer.

Another aspect of the disclosure relates to a fatty alcohol dehydration product prepared according to the processes described herein, according to the aspects of this disclosure. The dehydration product contains:
at least 85 mol % (such as at least 90 mol %, at least 95 mol %, at least 96 mol %, at least 97 mol %, at least 98 mol %, at least 99 mol %, or virtually 100 mol %) of a linear olefin (e.g., linear alpha olefin) or its isomer,
no more than about 5 mol % (such as no more than about 3 mol %, no more than about 2.5 mol %, no more than about 2 mol %, no more than about 1.5 mol %, no more than about 1.2 mol %, no more than about 1 mol %, no more than about 0.5 mol %, or no more than about 0.2 mol %) branched olefin, and/or
no more than about 5 mol % (such as no more than about 3 mol %, no more than about 2.5 mol %, no more than about 2 mol %, no more than about 1.6 mol %, no more than about 1.5 mol %, no more than about 1 mol %, or no more than about 0.5 mol %) ether.

Additional aspects, advantages and features of the disclosure are set forth in this specification, and in part will become apparent to those skilled in the art on examination of the following or may be learned by practice of the invention. The inventions disclosed in this application are not limited to any particular set of or combination of aspects, advantages, and features. It is contemplated that various combinations of the stated aspects, advantages and features make up the inventions disclosed in this application.

### BRIEF DESCRIPTION OF SEVERAL VIEWS OF THE DRAWING

FIG. 1 shows nitrogen adsorption-desorption isotherms of commercial and novel FER-type zeolite catalysts at 77K.

FIG. 2 shows powder X-ray diffraction (XRD) patterns of calcined FER samples.

FIG. 3 shows temperature-programmed desorption of ammonia (TPD-NH3) for FER samples.

FIG. 4 shows conversion of 2-propanol over various catalysts.

### DETAILED DESCRIPTION OF THE INVENTION

As used herein, unless indicated otherwise, the indefinite article “a” can mean “one” or “at least one.” For example, “a monomer” can mean “one monomer” or “at least one monomer.” Likewise, the definite article “the” can mean “one” or “at least one.” For example, “the monomer” can mean “one monomer” or “at least one monomer.”

The disclosure provides novel, modified ferrierite (FER)-type zeolite catalysts and their use in fatty alcohol dehydration processes. FER-type zeolites may be modified through property-enhancement treatments to create mesopores within the zeolite structure. These mesopores may increase the total pore volume and surface area of the zeolites, enhancing catalytic performance such as catalytic activity and selectivity toward desired products. Modified FER-type zeolites as described herein exhibit improved characteristics compared to unmodified, commercially available FER-type zeolites, such as the creation of mesopores in the zeolite structure (as opposed to zero or minimized mesopores in a conventional FER-type zeolite) thus an increase total pore volume, a higher surface area, and an enhanced catalytic activity and selectivity toward olefins particular linear olefins. These improvements make the modified zeolites particularly suitable for use as catalysts in fatty alcohol dehydration reactions.

### Modified Ferrierite (FER)-Type Zeolite

One aspect of the invention relates to a modified ferrierite (FER)-type zeolite. The modified ferrierite (FER)-type zeolite comprises a FER-type zeolite that has been modified by a property-enhancement treatment. The modified FER-type zeolite has not only micropores but also mesopores.

The term “zeolite” as described herein refers to crystalline aluminosilicate materials that are microporous and are formed from AlO₂ and SiO₂.

The catalytic performance of zeolites is closely related to their porosity characteristics and acidic properties. The acidic sites in zeolites can catalyze the dehydration process, but controlling the product distribution and minimizing byproduct formation often requires careful tuning of the catalyst properties. Factors such as acid site density, acid strength, and pore structure may significantly influence the catalytic behavior and product selectivity in these reactions.

Described herein are ways to modify an existing type of zeolite catalyst with enhanced porosity and acidity characteristics that address the limitations on low reaction efficiency and low product selectivity associated with conventional zeolite catalysts used in these catalytic reactions, such as fatty alcohol dehydrations.

There are different types of pores in zeolites depending the framework type of zeolites. For instance, micropores are those having an average pore-diameter of <2 nm; mesopores are those having an average pore-diameter of ≥2 nm (e.g., ranging from 2 nm to 50 nm); and macropores are those having an average pore-diameter of >50 nm.

A ferrierite (FER) type zeolite is a zeolite framework type that possess a two-dimensional pore network consisting of 10-membered ring channels intersecting with 8-membered ring channels. Conventional FER-type zeolites (the unmodified, commercially available FER-type zeolites) typically only contain micropores. While these micropores can impart molecular sieving effects, they may also impose diffusion limitations on reactants and products, potentially leading to reduced catalytic efficiency and rapid deactivation in some applications.

Conventional FER-type zeolites typically have zero or a small amount of mesopores. Theoretically, from zeolite structure topology point of view, any types of zeolites, including FER-type zeolite, would typically only contain micropores and not mesopores. During lab synthesis, however, occasionally but not always, synthetic zeolites made via conventional manners can have a small amount of mesopores due to crystal defects. Such a small amount of mesopores, however, is not significant enough to likely exhibit any property or performance advantage. This is illustrated in the catalyst characterization and performance evaluation of the comparative examples (MSZ35312 and CP914C) in Examples 3 and 4 below.

In this disclosure, FER-type zeolites have been modified to newly create significant amounts of mesopores in the zeolite structure. This improvement in the porosity of the zeolite catalyst in turn results in an enhanced overall pore volume and enhanced surface area of the zeolite. The presence of both micropores and mesopores in the modified FER-type zeolite may contribute to its enhanced properties compared to unmodified FER-type zeolites. The new creation of mesopores and significantly increased amounts of mesopore volume, which also leads to increase of accessible surface area and acid sites, in the modified FER-type zeolite may provide additional accessible surface area and facilitate mass transport within the zeolite structure, and result in an enhanced catalytic performance in various applications, such as fatty alcohol dehydration processes.

Accordingly, the modified FER-type zeolite may be characterized by its total mesopore volume. In some embodiments, the total mesopore volume of the modified FER-type zeolite may be greater than about 0.05 cm³/g. For example, the total mesopore volume of the modified FER-type zeolite may be about 0.06 cm³/g or greater, about 0.10 cm³/g or greater, or about 0.14 cm³/g or greater.

The modified FER-type zeolite may exhibit a significant increase in total mesopore volume compared to the unmodified FER-type zeolite. In some embodiments, the unmodified, conventional FER-type zeolite does not contain any mesopore in its zeolite structure, and the modification by the property-enhancement treatment newly creates mesopores in the zeolite structure. In some embodiments, the unmodified, conventional FER-type zeolite may contain a small amount of mesopores in its zeolite structure due to crystal defects, and the modified FER-type zeolite may have a total mesopore volume increase of at least about 50% compared to the same FER-type zeolite in its unmodified form. The increase in total mesopore volume may vary depending on the specific property-enhancement treatment applied to the FER-type zeolite. In some embodiments, the total mesopore volume increase may be at least about 100% compared to the same FER-type zeolite in its unmodified form. In some embodiments, the increase may be even more substantial, such as at least about 200%, at least about 300%, at least about 400%, at least about 500%, or at least about 600%, compared to the same FER-type zeolite in its unmodified form.

The modified FER-type zeolite may also be characterized by its total micropore volume. The micropores in the modified FER-type zeolite may help maintain the characteristic shape selectivity of the modified FER-type zeolites. In some embodiments, the total micropore volume of the modified FER-type zeolite range from about 0.10 to 0.30 cm³/g. For example, the total micropore volume of the modified FER-type zeolite may range from about 0.10 to about 0.25 cm³/g, from about 0.15 to about 0.25 cm³/g, from about 0.16 to about 0.22 cm³/g, or from about 0.16 to about 0.17 cm³/g.

The total micropore volume and/or mesopore volume of the modified FER-type zeolite may be measured by methods known in the art for measuring pore volumes in a solid catalyst. In some embodiments, the measurement of the total micropore volume and/or mesopore volume may be determined from an adsorption isotherm using a surface area and/or porosity analyzer. For instance, Ar adsorption isotherms obtained at 87K on a Surface Area and Porosity Analyzer may be used for such measurement, or N₂ adsorption isotherms obtained at 77k on Surface Area and Porosity Analyzer may be used. This measurement technique can provide accurate characterization of the pore structure of the modified FER-type zeolite.

In some embodiments, the micropore volume of the modified FER-type zeolite may be determined by a volumetric gas adsorption within the micropores, for instance, cm³ g⁻¹ at standard temperature and pressure. This may be estimated from the analysis of semi-logarithmic derivative plots of the adsorption isotherm using the equation (∂(V_{ads})/∂(ln (P/P₀)) vs. ln(P/P₀)) to identify the first maximum, representing the micropore filling transition, and subsequent minimum, representing the end of micropore filling.

In some embodiments, the mesopore volume of the modified FER-type zeolite may be obtained by a gas adsorption analysis of mesopores, e.g., by converting standard gas adsorption volumes (cm³ gcat⁻¹ at STP) to liquid volumes using a density conversion factor. In one embodiment, the liquid density of Ar at −186° C. (87K) may be used. In other embodiments, the liquid density of N₂ at 77K may be used.

The total mesopore volume increase may be determined by comparing the measurements of the modified FER-type zeolite against that of the same FER-type zeolite but in its unmodified form. These measurements may provide quantitative data on the pore structure changes resulting from the property-enhancement treatment.

The modified FER-type zeolite may be characterized by its surface area, such as Brunauer-Emmett-Teller (BET) surface area. In some embodiments, the modified FER-type zeolite may have a BET surface area of greater than about 290 m²/g. For instance, the BET surface area of the modified FER-type zeolite may be greater than about 300 m²/g, greater than about 310 m²/g, or greater than about 315 m²/g. In some embodiments, the modified FER-type zeolite may exhibit an even higher BET surface area, such as greater than about 350 m²/g, greater than about 360 m²/g, or greater than about 370 m²/g. In some embodiments, the measurement of the BET surface area may be determined from a gas molecule adsorption on the solid surface of the modified FER-type zeolite using a surface area and/or porosity analyzer. For instance, N₂ adsorption at 77K measured on a Surface Area and Porosity Analyzer may be used for such measurement.

The modified FER-type zeolite may exhibit a significant increase in BET surface area compared to the unmodified FER-type zeolites. In some embodiments, the modified FER-type zeolite may have a BET surface area increase of at least about 20% compared to the same FER-type zeolite in its unmodified form. The increase in BET surface area may vary depending on the specific property-enhancement treatment applied to the FER-type zeolite. In some embodiments, the BET surface area increase may be at least about 25% compared to the same FER-type zeolite in its unmodified form. In some embodiments, the increase may be even more substantial, such as at least about 30%, at least about 32%, or at least about 35% compared to the same FER-type zeolite in its unmodified form. This significant increase in BET surface area of the modified FER-type zeolite may be attributed to the creation of new and/or additional mesopores during the property-enhancement treatment. This increased surface area may contribute to the enhanced catalytic performance (e.g., providing additional active sites within the zeolite structure) in various applications, such as fatty alcohol dehydration processes.

The surface area (e.g., BET surface area) of the modified FER-type zeolite may be measured by methods known in the art for measuring surface area of a solid catalyst. In some embodiments, the measurement of the surface area (e.g., BET surface area) may be determined from an adsorption isotherm using a surface area analyzer. For instance, Ar adsorption isotherms obtained at 87K on a Surface Area and Porosity Analyzer, based on the BET theory, may be used for such measurement, or N₂ adsorption isotherms obtained at 77K. This measurement technique can provide accurate characterization of the surface area of the modified FER-type zeolite.

The BET surface area increase may be determined by comparing the measurements of the modified FER-type zeolite against that of the same FER-type zeolite but in its unmodified form. These measurements may provide quantitative data on the surface area changes resulting from the property-enhancement treatment.

The modified FER-type zeolite may comprise silicon oxide (SiO₂) and aluminum oxide (Al₂O₃). The relative amounts of silicon oxide and aluminum oxide in the zeolite structure may be characterized by the SiO₂:Al₂O₃ molar ratio. The SiO₂:Al₂O₃ molar ratio may influence various physical and chemical properties of the modified FER-type zeolite, such as acidity, pore size and structure, and thermal and hydrothermal stability. A lower SiO₂:Al₂O₃ ratio may result in a higher number of acid sites and a higher acidity, while a higher ratio may lead to a lower number of acid sites and a lower acidity. A higher SiO₂:Al₂O₃ ratio may also lead to increased stability of the modified FER-type zeolite, making the zeolite less susceptible to degradation or dissolution. Additionally, the process of creating mesoporosity for the modified FER-type zeolite can cause some loss of SiO₂ in the structure, resulting in a smaller SiO₂:Al₂O₃ ratio compared to that of the unmodified, commercially available FER-type zeolite from which the modified FER-type zeolite is prepared. That is to say, the modified FER-type zeolite has a SiO₂:Al₂O₃ ratio equal to or smaller than that of corresponding unmodified, commercially available FER-type zeolite. For instance, if the unmodified, commercially available FER-type zeolite used for modification has a SiO₂:Al₂O₃ ratio of 50, the modified FER-type zeolite can have a SiO₂:Al₂O₃ ratio of equal to or smaller than 50. In some embodiments, the SiO₂:Al₂O₃ molar ratio in the modified FER-type zeolite may be no greater than 60. This range of molar ratios may provide a balance of properties that make the FER-type zeolite suitable for various applications, including use as a catalyst in fatty alcohol dehydration processes. For instance, the SiO₂:Al₂O₃ molar ratio in the modified FER-type zeolite may be no greater than 50, no greater than 40, no greater than 35, or no greater than 20. The SiO₂:Al₂O₃ molar ratio of the modified FER-type zeolite may be determined through various analytical techniques, such as X-ray fluorescence (XRF) spectroscopy or inductively coupled plasma (ICP) analysis. These techniques may provide accurate measurements of the elemental composition of the zeolite.

The composition of the modified FER-type zeolite, including its SiO₂:Al₂O₃ molar ratio, may be controlled during the synthesis process. Factors such as the silicon and aluminum sources used, the synthesis temperature, and the pH of the synthesis mixture may influence the final composition of the zeolite.

The modified FER-type zeolite described herein may be prepared by modifying a FER-type zeolite through a property-enhancement treatment. This treatment may comprise mixing the FER-type zeolite with an aqueous source of hydroxide ions (OH) and a surfactant. This mixing step may create (or additionally produce) mesopores within the zeolite structure. The creation (or additional production) of the mesopores may contribute to the increased total mesopore volume and surface area of the modified FER-type zeolite.

Any aqueous source of hydroxide ions (OH⁻) known to one skilled in the art may be applicable herein, such as an alkali metal hydroxide (e.g., NaOH, KOH, or CsOH). In some embodiments, the aqueous source of OH may be sodium hydroxide (NaOH). NaOH may provide a readily available and cost-effective source of hydroxide ions for the treatment process, even more suitable for large-scale treatments. NaOH may offer several more advantages such as its easy dissolution in water to create solutions of various concentrations, and its strong basic nature to effectively promote the formation of mesopores within the zeolite structure.

The concentration of the aqueous OH-source (e.g., NaOH) used in the property-enhancement treatment may vary depending on the desired modification level. In some embodiments, the concentration of the aqueous OH-source (e.g., NaOH) may range from 0.1 to 3M, such as 0.1 to 1M, or 0.1 to 0.5 M. These ranges of concentrations may provide sufficient hydroxide ions to create mesopores within the zeolite structure while avoiding excessive degradation of the zeolite framework.

The amount of the OH-source solution (e.g., NaOH solution) (by volume) relative to the amount of the FER-type zeolite (by weight) used in the mixing step may range from 10 to 100. This amount ratio may ensure adequate contact between the hydroxide ions and the zeolite particles while maintaining a suitable mixture consistency for the treatment process.

In some embodiments, adjusting the concentration of the aqueous OH-source (e.g., NaOH) and the amount ratio of the aqueous OH-source (e.g., NaOH) to the FER-type zeolite may influence factors such as the total mesopore volume, surface area, and pore size distribution of the modified FER-type zeolite.

The surfactant used in the property-enhancement treatment may an ionic surfactant. Ionic surfactants may be particularly effective in modifying the surface of zeolites and creating mesopores within the modified FER-type zeolite structure to enhance the adsorption capacity of the zeolite. In some embodiments, the ionic surfactant may be a cationic surfactant. Cationic surfactants have a positively charged head and a hydrocarbon tail, which allows them to form a monolayer on the negatively charged surface of zeolite, thereby interacting strongly with the zeolite framework and leading to more effective mesopore formation during the property-enhancement treatment.

One type of cationic surfactant that may be used in the property-enhancement treatment is a salt of cetyltrimethylammonium (CTA) cation. CTA-based surfactants may be particularly effective due to their long hydrocarbon chain and positively charged quaternary ammonium group. In some embodiments, the CTA-based surfactant may be cetyltrimethylammonium bromide (CTAB) or cetyltrimethylammonium chloride (CTAC). CTAB may be a commonly available and effective cationic surfactant for creating mesopores in zeolite structures. CTAC may offer similar properties to CTAB and may be used as an alternative or in combination with CTAB. In some embodiments, a mixture of CTAB and CTAC may be used, which may offer advantages in terms of mesopore formation or distribution compared to using a single surfactant.

The amount of the surfactant used relative to the amount of the FER-type zeolite may be adjusted to achieve the desired modifications to the FER-type zeolite structure, based on factors such as the desired mesopore volume, the type of surfactant used, and the concentration of hydroxide ions in the treatment mixture. In some embodiments, the weight ratio between the amount of the surfactant and the amount of the FER-type zeolite may range from 0.2 to 1. This range may provide a sufficient amount of surfactant to create mesopores while avoiding excessive surfactant that could potentially block pores or hinder the treatment process.

In the mixing step of the property-enhancement treatment, the interaction between the surfactant molecules and the zeolite framework, facilitated by the presence of hydroxide ions, may lead to the creation of additional pore volume and surface area in the modified FER-type zeolite.

The property-enhancement treatment may further comprise heating the mixture of the FER-type zeolite, the aqueous source of hydroxide ions (OH⁻), and the surfactant. The heating step may facilitate the formation of mesopores within the zeolite structure. The heating step may be carried out at a temperature ranging from about 50° C. to about 120° C. For instance, the heating temperature may range from about 80° C. to about 120° C., from about 90° C. to about 110° C., or from about 90° C. to about 100° C. In one embodiment, the heating temperature may be about 95° C.

The combination of the mixing and heating steps in the property-enhancement treatment may result in a modified FER-type zeolite with increased mesopore volume and surface area compared to the same FER-type zeolite, unmodified.

The duration of the property-enhancement may vary depending on factors such as the desired mesopore volume, the specific FER-type zeolite being treated, and the concentration of hydroxide ions and surfactant in the mixture. Typically, the treatment may last over 1 hour or longer, such as 5 hours or longer, e.g., 5 to 48 hours. In some embodiments, the mixing step is carried out at room temperature for a short period of time (such as tens of minutes to 2 hour, e.g., about 30 minutes), followed by a heating step (e.g., hydrothermal treatment) for 5 hours or longer (e.g., 12 hours or longer).

The property-enhancement treatment may comprise additional steps to further modify the FER-type zeolite. In some embodiments, the additional steps may comprise separating solid products resulted from the property-enhancement treatment from the solution (e.g., through filtration), purifying the separated product (e.g., by washing with water), and/or drying (e.g., via heating to dry out water) the resulting product to obtain the modified FER-type zeolite.

In some embodiments, the additional steps may comprise calcining the modified FER-type zeolite. The calcining step may involve heating the modified FER-type zeolite at a temperature of about 400° C. or above. In some embodiments, the calcining temperature may be about 450° C. or above, about 500° C. or above, or about 550° C. or above. In one embodiment, the calcining temperature may be about 550° C. Calcining the modified FER-type zeolite at these elevated temperatures may serve multiple purposes. For instance, the high temperature may remove organic compounds, such as the surfactant used in the property-enhancement treatment, from the pores of the zeolite. This removal may open up the newly created mesopores, making them accessible for catalytic reactions. Additionally, the calcining step may help stabilize the modified zeolite structure. The duration of this calcination step may vary depending on factors such as the size of the zeolite particles, the heating temperature, and the amount of organic compounds in the zeolite. The calcining step may be carried out for a time sufficient to remove at least part of the surfactant from the zeolite, for instance, for 1 hour or longer, or 5 hours or longer.

In some embodiments, the additional steps may comprise converting the modified FER-type zeolite to a protonated form of the zeolite. The protonated form may exhibit enhanced catalytic activity due to the presence of acidic proton sites within the zeolite structure.

The conversion to a protonated form may be achieved through various methods. The modified FER-type zeolite may be converted to a protonated form through a multi-step process. In some embodiments, the converting step may involve an ion-exchange process followed by a thermal treatment.

In some embodiments, the modified FER-type zeolite may be subjected to an aqueous source of ammonium ions, such as an ammonium nitrate solution. This ion-exchange process may replace at least some metal ions in the zeolite with ammonium ions, forming an ammonium form of the zeolite. The duration and temperature of this treatment may vary depending on factors such as the concentration of the ammonium ion solution and the desired degree of ion exchange. The ion-exchange process may be repeated for more than once to ensure the complete replacement of the metal ions.

In some embodiments, following the ion-exchange process, the ammonium form of the zeolite may be heated and/or calcinated to produce the protonated form. In some embodiments, this heating/calcining step may be carried out at a temperature of about 300° C. or above. For example, the temperature may be about 350° C. or above, or about 400° C. or above. In one embodiment, the temperature may be about 400° C. The heating/calcining step may cause the ammonium ions to decompose, leaving behind protons at the acid sites within the zeolite structure. The duration of the heating or calcination step may vary depending on factors such as the size of the zeolite particles, the heating temperature, and the desired degree of protonation. In some embodiments, the heating/calcining step may be carried out for 1 hour or longer, or 5 hours or longer, to ensure complete conversion to the protonated form.

The protonated form of the modified FER-type zeolite may exhibit enhanced catalytic activity compared to the non-protonated form. The presence of acidic proton sites within the zeolite structure may contribute to its improved catalytic performance.

### Fatty Alcohol Dehydration Processes

The modified FER-type zeolites described herein may be used as a catalyst in various catalytic reactions, such as a fatty alcohol dehydration process. Accordingly, another aspect of the invention relates to a process for a fatty alcohol dehydration. The process comprises providing a feedstock containing a fatty alcohol, and dehydrating the fatty alcohol in the feedstock in the presence of the modified FER-type zeolite catalyst as described herein to obtain a dehydration product.

All above descriptions and all embodiments discussed in the above aspect relating to the modified FER-type zeolite, including its characterization by various properties such as mesopore and micropore volumes and surface areas; various property-enhancement treatment methods and steps; and various components and the amounts and treatment conditions used in the property-enhancement treatment methods and steps are all applicable to this aspect of the invention relating to a process for a fatty alcohol dehydration.

In some embodiments, the feedstock used in the fatty alcohol dehydration process may be a bio-based feedstock. Bio-based feedstocks may be derived from renewable sources, such as sugar, cellulose, plant oils or animal fats. The use of bio-based feedstocks in the fatty alcohol dehydration process may contribute to sustainability efforts and reduce reliance on petrochemical sources. In some embodiments, the bio-based feedstock may be obtained from agricultural waste products or byproducts. For example, fatty alcohols may be derived from agricultural waste, waste cooking oils or other lipid-rich waste streams. The use of such waste materials as feedstocks may provide additional environmental benefits by reducing waste and promoting circular economy principles.

The fatty alcohol in the feedstock may be derived from various sources via well-known technologies, such as a fermentation, solvent extraction, and/or transesterification-hydrogenation technology. The choice of fatty alcohol source may depend on factors such as availability, cost, and desired product characteristics. In some embodiments, a combination of sources may be used to obtain fatty alcohols with specific chain length distributions or properties.

In some embodiments, the fatty alcohol may be derived from fats. Fats, such as animal fats (such as tallow or fish oil) or vegetable fats, may be processed through hydrolysis and hydrogenation to produce fatty alcohols of varying chain lengths. In some embodiments, the fatty alcohols may be derived from waxes. Waxes, which may be of plant or animal origin, may contain fatty alcohols or fatty acid esters that can be converted to fatty alcohols through chemical processes such as hydrogenation. In some embodiments, the fatty alcohol may be derived from plant oils (such as coconut oil, palm kernel oil, or rapeseed oil). These oils may undergo processes such as transesterification and hydrogenation to yield fatty alcohols.

In some embodiments, the fatty alcohol may be derived from petrochemical sources. In some embodiments, petrochemical-derived fatty alcohols may be synthesized through processes such as the Ziegler process or oxo synthesis, using ethylene or other olefins as starting materials.

The source of the fatty alcohol may influence its chemical composition and properties, which may in turn affect the dehydration process and the resulting products.

The fatty alcohol used in the dehydration process may have various characteristics. In some embodiments, the fatty alcohol may comprise one or more OH groups attached to an aliphatic chain of C₄-C₂₆ carbon atoms. (e.g., the aliphatic chain may have C₆-C₂₄ carbon atoms, C₆-C₂₂ carbon atoms, or C₄-C₈ carbon atoms). In some embodiments, fatty alcohols with the aliphatic chain of C₆-C₂₂ carbon atoms may be desirable because they may be commonly derived from natural sources such as plant oils or animal fats. In some embodiments, fatty alcohols with a shorter-chain, an aliphatic chain of C₄-C₈ carbon atoms, may be desirable as they may be useful for producing shorter-chain olefins through the dehydration process. In other embodiments, the fatty alcohol may comprise one or more OH groups attached to an aliphatic chain of C₂-C₂₆ carbon atoms, or of C₃-C₂₆ carbon atoms (e.g., an aliphatic chain of C₂-C₂₄ carbon atoms, C₃-C₂₂ carbon atoms, or C₄-C₈ carbon atoms). Thus, as used herein, the term “fatty alcohol” can include ethanol and propanol (including any isomer thereof).

In some embodiments, the fatty alcohol may be a C₄-C₈ alcohol. For example, the fatty alcohol may be propanol (isomers such as n-propanol and isopropanol), or butanol (in various isomeric forms such as n-butanol, sec-butanol, isobutanol, and tert-butanol), pentanol (in various isomeric forms including n-pentanol and isopentanol), hexanol (in various isomeric forms, including 1-hexanol), heptanol (in various isomeric forms, including n-heptanol), octanol (in several isomeric forms, including 1-octanol). In embodiments, it can be any combination of these fatty alcohols. In some embodiments, the C₄-C₈ alcohols may be derived from bio-based sources. For example, butanol may be produced through fermentation processes using biomass feedstocks. In other embodiments, the C₄-C₈ alcohols may be synthesized from petrochemical sources. In other embodiments, the C₃-C₈ alcohols may be synthesized from petrochemical sources.

The fatty alcohol may have one or more specific characteristics, including the number of carbon atoms in the aliphatic chain, the number and position of OH groups, and the structure of the aliphatic chain, selection of which may influence the dehydration process and the properties of the resulting olefin products. The fatty alcohol may comprise any one or a combination of two or more of the following characteristics:
(i) Chain type. In some embodiments, the fatty alcohol may have (a) a linear chain. In other embodiments, the fatty alcohol may have (b) a branched chain. Linear chain fatty alcohols may promote the production of linear olefins upon dehydration, whereas fatty alcohol having a branched chain may promote the production of branched olefins upon dehydration.
(ii) Number of OH groups. In some embodiments, the fatty alcohol may have (a) a single OH group. For instance, the fatty alcohols may be primary alcohols with a single OH group attached to a terminal carbon atom, or secondary alcohols with a single OH group attached to a non-terminal carbon atom. In other embodiments, the fatty alcohol may be polyhydric fatty alcohols having (b) two or more OH groups. The number of OH groups may affect the reactivity and dehydration behavior of the fatty alcohol.
(iii) Position of OH group. In some embodiments, the fatty alcohol may be (a) a primary alcohol having an OH group attached to the terminal carbon atom. In other embodiments, the fatty alcohol may be (b) a secondary alcohol having an OH group attached to a non-terminal carbon atom. The position of the OH group may influence the dehydration pathway and product distribution.
(iv) Saturation level. In some embodiments, the fatty alcohol may have (a) a saturated chain, containing only single bonds between carbon atoms. In other embodiments, the fatty alcohol may have (b) an unsaturated chain, containing one or more double bonds between carbon atoms. The saturation level may affect the stability and reactivity of the fatty alcohol during the dehydration process.

In some embodiments, the fatty alcohol may have a combination of different characteristics from different groups discussed above. In some embodiments, the fatty alcohol may have a linear, saturated chain. For example, the fatty alcohol may be a linear, saturated chain primary alcohol having a single OH group attached to the terminal carbon atom. This structure may promote the formation of terminal olefins (alpha-olefins) upon dehydration. In some embodiments, the dehydration product comprises a linear olefin, such as linear alpha olefin, which may be valuable intermediates in various industrial processes. In other embodiments, the dehydration product may comprise at least one of an alpha-olefin, an internal olefin, a branched olefin, or a mixture thereof. As used herein, “internal olefin” can mean an olefin having a non-terminal double bond, such as, e.g., 2-butene (CH₃—CH═CH—CH₃). The branched olefin can result from a dehydration of a branched alcohol, such as 2-butanol.

The dehydration process may be carried out under various conditions. The use of linear, saturated chain fatty alcohols in the dehydration process may influence the catalyst selection and reaction conditions. In some embodiments, the modified FER-type zeolite catalyst may be particularly effective for dehydrating linear, saturated chain fatty alcohols. The mesoporous structure of the modified FER-type zeolite catalyst may facilitate the diffusion of these linear molecules, potentially improving the reaction rates and product selectivity.

In some embodiments, the dehydrating step may be carried out in the presence of a solvent. In some embodiments, the solvent may act as a diluent, helping to control the concentration of reactants and products in the reaction mixture, and helping to prevent undesired side reactions or product degradation. In some embodiments, the solvent may facilitate heat transfer within the reaction mixture, helping maintain a more uniform temperature throughout the reaction vessel, potentially leading to more consistent reaction conditions. In some embodiments, the solvent may also aid in the dissolution of the fatty alcohol feedstock and the dispersion of the catalyst particles, enhancing the contact between the reactants and the catalyst, potentially improving reaction rates and efficiency. The choice of solvent may depend on various factors, including the specific fatty alcohol being dehydrated, the desired reaction conditions, and the ease of product separation. In some embodiments, the solvent may be selected based on its boiling point to facilitate product recovery through distillation. The amount of solvent used in the dehydration process may vary depending on the specific reaction conditions and desired outcomes.

In some embodiments, the solvent used in the dehydration process may be a hydrocarbon solvent. Exemplary hydrocarbon solvents that may be used in the dehydration process include, but are not limited to dodecane, hexane, cyclohexane, and toluene. In one embodiment, the solvent is dodecane.

In some embodiments, the solvent may be recovered and recycled after the dehydration process. This recycling may improve the overall efficiency and sustainability of the process, potentially reducing waste and operational costs.

The dehydrating step may be carried out under one or more of specific reaction conditions to promote efficient conversion of the fatty alcohol to desired olefin products. These conditions may comprise:
a weight ratio of catalyst to fatty alcohol of 0.05-0.15 (e.g., about 0.1); and/or
a reaction temperature ranging from about 200° C. to about 300° C. (e.g., from about 200° C. to about 270° C., from about 210° C. to about 230° C., or at about 220° C.); and/or
a stirring speed ranging from 250 to 1000 rpm (e.g., from 300 to 600 rpm or at about 500 rpm).

In some embodiments, the weight ratio of the modified FER-type zeolite catalyst to the fatty alcohol may range from 0.05 to 0.15. This ratio may influence the reaction kinetics and product distribution. In some embodiments, the weight ratio of catalyst to fatty alcohol may be about 0.1.

In some embodiments, the reaction temperature may range from about 200° C. to about 300° C., or from about 200° C. to about 500° C. This temperature range may provide sufficient energy for the dehydration reaction while avoiding excessive thermal degradation of the reactants or products. In some embodiments, the reaction temperature may range from about 200° C. to about 270° C., such as from about 210° C. to about 230° C. In one embodiment, the reaction temperature may be about 220° C. This specific temperature may be selected based on factors such as the boiling point of the fatty alcohol, the thermal stability of the catalyst, and the desired reaction rate.

In some embodiments, the stirring speed may range from 250 to 1000 rpm. This range of stirring speeds may provide adequate mixing while avoiding excessive mechanical stress on the catalyst particles. In some embodiments, the stirring speed may range from 300 to 600 rpm. In one embodiment, the stirring speed may be about 500 rpm. In another embodiment, the stirring speed may be about 700 rpm.

The dehydration process using the modified FER-type zeolite catalyst may result in an improved reaction rate compared to a dehydration process carried out under the otherwise same conditions, except for using an unmodified FER-type zeolite. In some embodiments, this improved reaction rate may be characterized by a higher site time yield under mild reaction conditions. Site time yield, also known as turnover rate, may be a measure of catalytic activity that represents the number of reactant molecules converted per catalytic site per unit time. In the context of fatty alcohol dehydration, the site time yield may indicate how efficiently the modified FER-type zeolite catalyst converts fatty alcohol molecules to olefin products.

In some embodiments, the dehydration process using the modified FER-type zeolite may achieve a site time yield that is at least 10% (e.g., at least 15%, at least 20%, or at least 25%) higher compared to the site time yield of a dehydration reaction carried out under the same conditions but in the presence of the same FER-type zeolite, unmodified. In some embodiments, the improvement in site time yield may be even more significant. For instance, the site time yield may be at least 30% higher, at least 35% higher, at least 40% higher, at least 45% higher, at least 50% higher, at least 60% higher, at least 70% higher, at least 80% higher, at least 90% higher, or at least 100% higher. In some embodiments, the improvement in site time yield may be at least 30% higher, at least 35% higher, at least 40% higher, or at least 45% higher compared to the site time yield of a dehydration reaction carried out under the same conditions but in the presence of the same FER-type zeolite, unmodified.

The substantial improvements in reaction rate and site time yield may be attributed to the modified FER-type zeolite used herein. In some embodiments, the increased mesopore volume and surface area resulting from the property-enhancement treatment may provide more accessible active sites for the fatty alcohol dehydration reaction. The presence of mesopores may also facilitate faster diffusion of reactants and products within the zeolite structure, potentially contributing to the higher site time yield. In some embodiments, the property-enhancement treatment used to modify the FER-type zeolite may alter the acid site distribution or strength, which may influence the catalytic activity.

The higher site time yield achieved by the modified FER-type zeolite may offer several potential advantages in industrial applications, such as an enhanced catalytic activity allowing for a higher throughput in dehydration processes to increase production efficiency and reduce processing times.

The dehydration process using the modified FER-type zeolite catalyst may result in an improved selectivity towards linear olefins or their isomers. In some embodiments, the dehydrating step may have a selectivity toward a linear olefin or its isomer of at least 85 mol %. The selectivity may be even higher in some cases, such as at least 90 mol %, at least 95 mol %, at least 96 mol %, at least 97 mol %, at least 98 mol %, at least 99 mol %, or virtually 100 mol %. This high selectivity may indicate that the modified FER-type zeolite catalyst effectively promotes the formation of desired linear olefin products while minimizing unwanted byproducts. The high selectivity towards linear olefins or their isomers may be attributed to various factors, such as enhanced porosity properties (i.e., creating and enhancing the mesoporosity) and enhanced acid functions. For instance, the presence of mesoporous structure and the significant creation of mesopore volumes in the modified FER-type zeolite may facilitate the diffusion of reactants and desirable products (i.e., linear olefins), reducing the occurrence of secondary reactions that could lead to the formation of the byproducts (e.g., branched or cyclic olefin; or ether formation that occurs when reactants/products are trapped within the pores of microporous catalyst).

The improved selectivity may be characterized by comparing the process using the modified FER-type zeolite to a process using an unmodified FER-type zeolite under the otherwise same reaction conditions. In some embodiments, the selectivity toward a linear olefin or its isomer may be improved by at least 5% compared to a dehydration reaction carried out under the same conditions but in the presence of the same FER-type zeolite, unmodified. The improvement in selectivity may be even more significant in some cases, such as at least 10%, at least 15%, at least 20%, at least 25%, or at least 30%.

Besides linear olefins, the dehydration of a fatty alcohol can also produce various byproducts, such as branched olefins, oligomers, and ethers. Using the modified FER-type zeolite catalyst may also result in reduced formation of byproducts for the reasons discussed above when discussing using the modified FER-type zeolite catalyst to obtain an improved selectivity towards linear olefins or their isomers.

In some embodiments, the dehydration product may contain a reduced amount of a byproduct, such as a branched olefin, an ether, and/or an oligomer. The amount of byproduct may be reduced by at least 50% compared to a dehydration product produced under the same conditions but in the presence of the same FER-type zeolite, unmodified. In some embodiments, the reduction in byproduct formation may be even more significant, such as at least 60%, at least 65%, at least 70%, at least 75%, at least 80%, at least 85%, or at least 90%.

The high selectivity towards linear olefins or their isomers and other derivatives, and reduced byproduct formation may be determined through various analytical techniques, such as gas chromatography or certain spectroscopic methods, to identify and quantify various components (e.g., the linear olefin content and other byproduct contents) in the product mixture. These analytical methods may provide detailed information on the product distribution and the extent of selectivity achieved in the dehydration process.

The ability to achieve high selectivity towards linear olefins or their isomers and significant reductions in byproduct formation may offer several advantages in industrial applications, such as improved product purity that may simplify downstream separation and purification processes, and better and more efficient conversion the feedstock materials to the desired linear olefin products.

One example of fatty alcohol dehydration process uses 1-hexanol as the fatty alcohol. The dehydration of 1-hexanol using the modified FER-type zeolite catalyst may result in a C₆ linear olefin or its isomer as the primary dehydration product. The dehydration of 1-hexanol using the modified FER-type zeolite catalyst exhibits high reaction rate characterized by a high site time yield, a high selectivity towards C₆ linear olefins or their isomers, and a reduced amount of a byproduct. The C₆ linear olefins or their isomers produced from 1-hexanol dehydration may comprise trans-2-hexene, cis-2-hexene, trans-3-hexene, and/or cis-3-hexene. The dehydration of 1-hexanol may produce small amounts of byproducts such as branched C₆ olefins (e.g., 4-methyl-1-pentene) or ether (e.g., di-n-hexyl ether may be produced through the condensation of two 1-hexanol molecules).

In some embodiments, by using the modified FER-type zeolite catalyst in the dehydration of 1-hexanol, the selectivity toward a C₆ linear olefin or its isomer may be at least 85 mol %. The selectivity may be even higher in some instances, such as at least 90 mol %, at least 95 mol %, at least 96 mol %, at least 97 mol %, at least 98 mol %, at least 99 mol %, or virtually 100 mol %. In some embodiments, the dehydration of 1-hexanol using the modified FER-type zeolite catalyst may show an improved selectivity toward a C₆ linear olefin or its isomer by at least 5% compared to a dehydration reaction carried out under the same conditions but in the presence of the same FER-type zeolite, unmodified. In some embodiments, the improvement in selectivity may be at least 10%, at least 15%, at least 20%, at least 25%, or at least 30%.

In some embodiments, the dehydration of 1-hexanol using the modified FER-type zeolite catalyst may have a high site time yield. For instance, the site time yield under mild reaction conditions may be at 2.4 kg_{reactant}kg⁻¹_{catalyst}hr⁻¹ or above, or at about 2.5 kg_{reactant}kg⁻¹_{catalyst}hr⁻¹ or above. In other embodiments, the site time yield may reach about 3.0 kg_{reactant}kg⁻¹_{catalyst}hr⁻¹ or above, about 3.1 kg_{reactant}kg⁻¹_{catalyst}hr⁻¹ or above, about 3.2 kg_{reactant}kg⁻¹_{catalyst}hr⁻¹ or above, or about 3.5 kg_{reactant}kg⁻¹_{catalyst}hr⁻¹ or above.

In some embodiments, by using the modified FER-type zeolite catalyst in the dehydration of 1-hexanol, the amount of byproduct may be reduced by at least 50% compared to a dehydration reaction carried out under the same conditions but in the presence of the same FER-type zeolite, unmodified. The reduction in byproduct formation may be even more significant in some instances, such as at least 60%, at least 65%, at least 70%, at least 75%, at least 80%, at least 85%, or at least 90%. The reduction of these byproducts may contribute to improved product purity and process efficiency.

In all the above embodiments relating to the reaction rate, the mild reaction conditions for achieving these site time yields may comprise a reaction temperature ranging from about 200° C. to about 300° C. In some embodiments, the temperature range may be from about 200° C. to about 270° C., from about 210° C. to about 230° C., or about 220° C.

The fatty alcohol dehydration process described herein using the modified FER-type zeolite catalyst may produce a dehydration product with specific composition, which may vary depending on factors such as the fatty alcohol used, the reaction conditions, and the specific properties of the modified FER-type zeolite catalyst. The ability to produce a dehydration product with high selectivity towards desired linear olefins and reduced byproduct formation may make the process using the modified FER-type zeolite catalyst particularly valuable for industrial applications.

In some embodiments, the fatty alcohol dehydration process described herein using the modified FER-type zeolite catalyst may produce a dehydration product containing at least 85 mol %, for instance, at least 90 mol %, at least 95 mol %, at least 96 mol %, at least 97 mol %, at least 98 mol %, at least 99 mol %, or virtually 100 mol % of a linear olefin (e.g., linear alpha olefin) or its isomer. In some embodiments, the dehydration product may contain no more than about 5 mol % branched olefin. In some embodiments, the content of branched olefin may be even lower, such as no more than about 3 mol %, no more than about 2.5 mol %, no more than about 2 mol %, no more than about 1.5 mol %, no more than about 1.2 mol %, no more than about 1 mol %, no more than about 0.5 mol %, or no more than about 0.2 mol %. In some embodiments, the dehydration product may contain no more than about 5 mol % ether. In some embodiments, the ether content may be further reduced, such as no more than about 3 mol %, no more than about 2.5 mol %, no more than about 2 mol %, no more than about 1.6 mol %, no more than about 1.5 mol %, no more than about 1 mol %, or no more than about 0.5 mol %.

The dehydration products obtained from linear, saturated chain fatty alcohols may have various applications. In some embodiments, the linear olefins produced may be used as intermediates in the production of detergents, lubricants, or plasticizers.

### EXAMPLES

The following examples are for illustrative purposes only and are not intended to limit, in any way, the scope of the present invention.

### Example 1—Synthesis of an Exemplary Modified Ferrierite (FER)-Type Zeolite

6 grams of MSZ35312 zeolite powders (a proton-form of FER-type zeolite, silica-to-alumina ratio of 50, ACS Material) were mixed with 80 mL of 0.4M NaOH solution and 3 grams of cetyltrimethylammonium bromide (CTAB). The mixture was stirred at room temperature for 30 minutes and then underwent hydrothermal treatment at 95° C. for 12 hours. The resulting solid product was separated from the solution through filtration, washed with deionized water, and dried at 95° C. for 3 hours. The dried sample was then calcined in a muffle furnace in the presence of air, heated to 550° C. at a rate of 1.5° C./minute, and held for 5 hours to remove the organic species inside the zeolite framework structure.

Afterward, the products were treated with 60 mL of 2N ammonium nitrate solution at 95° C. for 2 hours. The solution was cooled, decanted, and dried at 95° C. This process was repeated three times. The powders were then calcined again in a muffle furnace in the presence of air, heated to 400° C. at a rate of 2° C./minute, and held for 3 hours to convert the zeolite sample from an ammonium form to a proton form.

### Example 2—Synthesis of an Exemplary Modified FER-Type Zeolite

10 grams of CP914C zeolite powders (an ammonium-form of FER-type zeolite, silica-to-alumina ratio of 20, Zeolyst) were mixed with 100 mL of 0.2M NaOH solution and 5 grams of CTAB. The mixture was stirred at room temperature for 30 minutes and then underwent hydrothermal treatment at 95° C. for 12 hours. The resulting solid product was separated from the solution through filtration, washed with deionized water, and dried at 95° C. for 3 hours. The dried sample was then calcined in a muffle furnace in the presence of air, heated to 550° C. at a rate of 1.5° C./minute, and held for 5 hours to remove the organic species inside the zeolite framework structure.

Afterward, the products were treated with 100 mL of 2N ammonium nitrate solution at 95° C. for 2 hours. The solution was cooled, decanted, and dried at 95° C. This process was repeated three times. The powders were then calcined again in a muffle furnace in the presence of air, heated to 400° C. at a rate of 2° C./minute, and held for 3 hours to convert the zeolite sample from an ammonium form to a proton form.

### Example 3—Characterization of the Exemplary Modified FER-Type Zeolites BET

The micropore volumes and mesopore volumes of the exemplary modified FER-type zeolite catalysts, prepared according to Examples 1 and 2, as compared to those of the commercial FER-type zeolites, unmodified (MSZ35312 and CP914C), were determined from N₂ adsorption isotherms measured at 77 K on a Micromeritics ASAP 2020 Surface Area and Porosity Analyzer (ASAP 2020 Plus, Micromeritics Instrument Corporation). Typically, 0.03-0.05 g of pelleted and sieved sample (with a nominal diameter between 180-250 μm) were degassed by heating to 120° C. (at 10° C./minute) under vacuum (<5 μmHg) for 2 hours, further heating to 350° C. (at 10° C./minute) under vacuum (<5 μmHg), and holding for 9 hours. Volumetric gas adsorption within the micropores (cm³ g⁻¹ at standard temperature and pressure, STP) was estimated from the analysis of semi-logarithmic derivative plots of the adsorption isotherm (∂(V_{ads})/∂(ln(P/P₀)) vs. ln(P/P₀)) to identify the first maximum, representing the micropore filling transition, and the subsequent minimum, representing the end of micropore filling. Micropore volumes (cm³ g⁻¹) were obtained by converting standard gas adsorption volumes (cm³ gcat⁻¹ at STP) to liquid volumes using a density conversion factor assuming the liquid density of N₂ at 77K.

The Brunauer-Emmett-Teller (BET) surface area of the modified FER-type zeolite was performed using nitrogen gas (N2) as the adsorbate at 77 K due to its high affinity for solid surfaces on a Micromeritics ASAP 2020 Surface Area and Porosity Analyzer (ASAP 2020 Plus, Micromeritics Instrument Corporation), using a similar preparation method described above. The gas was introduced at low pressures and molecules began adsorbing to the surface. As gas pressure increased the monolayer was formed, followed by multilayer adsorption. The amount adsorbed was determined to calculate the surface area using the BET equation. The BET surface area was calculated from the monolayer capacity which is the volume of the first single layer of gas molecules or atoms adsorbed on the surface.

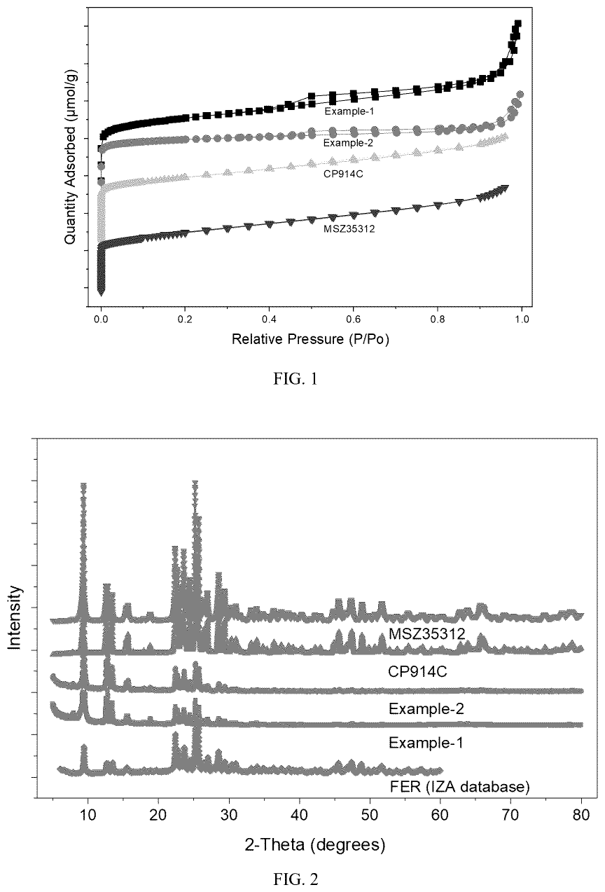

Nitrogen adsorption-desorption isotherms of commercial and novel FER-type zeolite catalysts is shown in FIG. 1. Compared to the parent material, Example-1 and Example-2 exhibit a distinct adsorption-desorption hysteresis loop at intermediate to high relative pressures. This indicates the successful generation of mesoporosity after the treatment. Because of the significantly new amounts of mesopores created in the modified FER-type catalysts, the surface area of the modified FER-type catalysts have also significantly increased. As shown in Table 1, the BET surface area of the modified FER-type zeolite was greater than about 290 m²/g (e.g., 317-375 m²/g), significantly higher than that of the unmodified commercial FER-type zeolite which was less than 290 m²/g (MSZ35312, 283 m²/g; CP914C, 252 m²/g).

TABLE 1

| Zeolite | | Mesopore | Micropore |
|---|---|---|---|
| catalyst | BET Surface Area | (>=2 nm) | (<2 nm) |
| sample | (m² g⁻¹) | Volume (cm³ g⁻¹) | volume (cm³ g−¹) |
| Example 1 | 375 | 0.14 | 0.16 |
| Example 2 | 317 | 0.10 | 0.17 |
| MSZ35312 | 283 | 0.02 | 0.17 |
| CP914C | 252 | 0.05 | 0.16 |

The unmodified commercial FER-type zeolite typically has only micropores and has no mesopores or very small amounts of mesopores. As indicated in Table 1, both unmodified commercial FER-type zeolite catalysts have minimized amounts of mesopores, with a total mesopore volume no greater than 0.05 cm³/g (MSZ35312, 0.02; CP914C, 0.05).

On the other hand, the property-enhancement treatment disclosed herein produced a novel modified FER-type zeolite catalyst that have significantly amounts of mesopores introduced into the zeolite structure, characterized by a significant increase in the total mesopore volume (e.g., 0.1-0.14 cm³ g⁻¹) relative to the unmodified commercial FER-type zeolites (e.g., <=0.05 cm³ g⁻¹).

The micropore and mesopore volumes (cm³ g⁻¹) were obtained by converting standard gas adsorption volumes (cm³ gcat⁻¹ at STP) to liquid volumes using a density conversion factor assuming the liquid density of Ar at −186° C. or assuming the liquid density of N₂ at 77K, using the built-in ASAP 2020 Software for calculating the volumes.

### XRD

The crystal structures of the Example-1 and Example-2 were characterized with powder X-ray diffraction (XRD) on a BRUKER AXS X-ray diffractometer using the Ni-filtered Cu KB radiation source at 40 kV and 20 mA. The crystal structures of the CP914C and MSZ35312 were characterized with powder X-ray diffraction (XRD) on a Malvern Panalytical X-ray diffractometer using the Ni-filtered Cu Kβ radiation source at 40 kV and 20 mA.

After calcination, samples are analyzed by powder X-ray diffraction (XRD). FIG. 2 shows the powder XRD patterns of the calcinated FER of Example 1, Example 2, Zeolyst and ACS Material. As depicted in FIG. 1, the powder XRD pattern of the calcinated products matched the typical phase of FER features from IZA database.

### Acidity Measurement

Temperature-programmed ammonia desorption (NH₃-TPD) analyses were performed using Micromeritics AutoChem III equipment. Typically, around 150 mg of samples were loaded in a U-tube quartz reactor and pretreated with helium flow of 50 mL/min. The sample was heated to 500° C. and then kept at 500° C. for 30 min. Then the sample was cooled to 110° C. under helium. After pretreatment, ammonia adsorption began with a mixture of ammonia (~10%) in helium at a flow rate of 50 mL/min for 30 min. To purge excess ammonia from the reaction environment, 50 mL/min of helium was passed through the bed until the baseline is stable. The desorption profiles were obtained under helium flow through the bed, in the range of 100° C. to 500° C., with a ramp of 20° C./min.

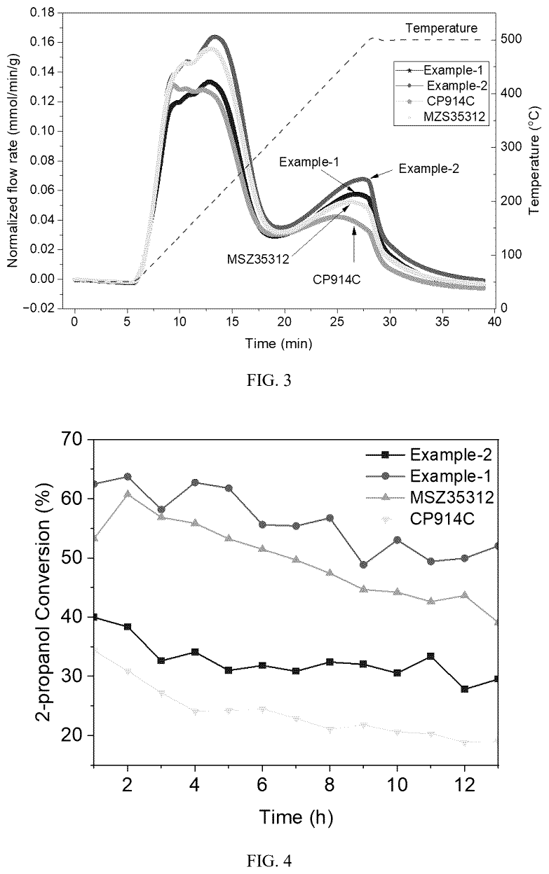

All four samples show NH3 desorption peaks from 5 min to 38 min (Peak I), and from 19.6 min to 38 min (Peak II) as shown in FIG. 3. Peak I correspond to weak acidity sites on FER samples and peak II corresponds to medium and strong acid sites. The NH3 quantity for each peak is shown in TABLE 2. Example-1 and Example-2 has more medium and strong acidity sites (0.52 and 0.59 mmol/g, respectively) than samples from ACS Material and Zeolyst (0.50 and 0.45 mmol/g, respectively). The higher amount of medium and strong acidity sites can result from the presence of the mesoporosity of the sample after the hydrothermal treatment.

TABLE 2
NH3 quantity from NH3-TPD

| | | | Total NH3 |
|---|---|---|---|
| | NH3 quantity from | NH3 quantity from | quantity |
| Sample | peak I (mmol/g) | peak II (mmol/g) | (mmol/g) |
| MSZ35312 (ACS | 1.37 | 0.50 | 1.87 |
| Material) | | | |
| CP914C (Zeolyst) | 1.13 | 0.45 | 1.57 |
| Example-1 | 1.12 | 0.52 | 1.64 |
| Example-2 | 1.43 | 0.59 | 2.02 |

Baseline started from 5 min to 38 min. Peak I is from time of 5.4 min to 19.6 min. Peak II is from time of 19.6 min to 38 min.

### Example 4—an Exemplary Fatty Alcohol Dehydration Employing the Exemplary Modified FER-Type Zeolites, e.g., 1-Hexanol Dehydration

The exemplary modified FER-type zeolite catalysts, prepared according to Examples 1 and 2, were employed to carry out a fatty alcohol dehydration reaction (e.g., dehydration of 1-hexanol) in this example. The results were compared to the fatty alcohol dehydration reactions carried out in presence of unmodified commercial FER-type zeolite catalysts (MSZ35312 and CP914C).

1-hexanol dehydration catalyst testing was performed in a 45 mL batch reactor (Parr Instruments 5000 series multiple reaction system) for each of the zeolite catalyst sample. The reactor was loaded with the catalyst sample (0.020 to 0.05 g) and 1-hexanol (0.5 g) dissolved in a solvent consisting of 10 mL of dodecane. The reactor was sealed, purged with 1000 psi of helium three times, at ambient temperature. The reactor was then heated to 220° C. at an initial pressure of 1 bar helium. Once the desired temperature was attained, usually within 25 minutes, the stirring speed was set to 500 rpm, signifying the start of reaction. Following the fixed-time batch run for 3 hours, the heating and stirring were simultaneously stopped. The reactor was allowed to cool in air, then the product samples were taken out and a known amount of internal standard (n-heptane) was added. The product samples filtered through a polyethersulfone syringe membrane with 0.2 μm pore size to separate the catalyst particles in the liquid samples. These liquid samples were analyzed by an Agilent Technologies 8890 gas chromatograph equipped with a flame ionization detector (FID). All the samples were injected automatically by an Agilent Technologies 7693 Series auto-injector with a 10 μL syringe. A CYCLODEX-B capillary column (30 m×0.25 mm I.D.×0.25 μm film thickness) was used for separation of different species. The diffusion limitations were eliminated in this study when the stirring speed is 700 rpm, and the carbon mass balance was closed at about 85%.

The reaction conditions for dehydration reaction employing each zeolite catalyst are: 50 mg zeolite catalyst; 0.5 g 1-hexanol; 10 mL dodecane (solvent); at 500 rpm rotation rate; with a reaction temperature of 220° C.; and reacted for 3 hours. The results are shown in Table 3.

TABLE 3
Dehydration of 1-hexanol over exemplified FER-
type zeolite catalysts, as compared to unmodified
commercial FER-type zeolite catalysts.

| Dehydration reaction | | | | |
|---|---|---|---|---|
| parameters | Example 1 | Example 2 | MSZ35312 | CP914C |
| Conversion (%) | 93.3 | 92.9 | 64.6 | 67.9 |
| Site time yield | 3.11 | 3.09 | 2.15 | 2.26 |
| (kg_{reactant}kg⁻¹_{catalyst}hr⁻¹) | | | | |
| Selectivity (mol %) | | | | |
| Linear olefins (1- | 98.2 | 97.2 | 84.2 | 91.7 |
| hexene + trans-2- | | | | |
| hexene + cis-2- | | | | |
| hexene + trans-3- | | | | |
| hexene + cis-3-hexene) | | | | |
| Branched olefins | 0.2 | 1.2 | 1 | 0.6 |
| (4-methyl-1-pentene) | | | | |
| di-n-hexyl ether | 1.6 | 1.6 | 14.8 | 7.7 |

The reaction conditions for dehydration reaction employing each zeolite catalyst are: 20 mg zeolite catalyst; 0.5 g 1-hexanol; 10 mL dodecane (solvent); at 700 rpm rotation rate; with a reaction temperature of 220° C.; and reacted for 3 hours. The results are shown in Table 3a.

TABLE 3a

| | Example-1 | Example-2 | | |
|---|---|---|---|---|
| | (working | (working | MSZ35312 | CP914C |
| | example) | example) | (ACS Material) | (Zeolyst) |
| Conversion (%) | 82.8 | 80.8 | 68.1 | 62 |
| Site time yield | 6.9 | 6.7 | 5.7 | 5.2 |
| (kg_{reactant} | | | | |
| kg⁻¹_{catalyst}hr⁻¹) | | | | |
| Selectivity (mol %) | | | | |
| 1-hexene | 12.2 | 12.5 | 13.2 | 12.9 |
| Linear olefins | 96.2 | 98.5 | 96 | 97.3 |
| (1-hexene + trans- | | | | |
| 2-hexene + cis-2- | | | | |
| hexene + trans-3- | | | | |
| hexene + cis-3- | | | | |
| hexene) | | | | |
| Branched olefins | 3.5 | 0.7 | 0.5 | 0.4 |
| (4-methyl-1- | | | | |
| pentene) | | | | |
| di-n-hexyl ether | 0.3 | 0.8 | 3.5 | 2.3 |

Without being bound by theory, dehydration of 1-hexanol typically involves several steps. First, 1-hexanol adsorbs onto the acidic sites of a zeolite and becomes protonated, forming an oxonium ion (protonated alcohol). The oxonium ion then loses a water molecule, creating a hexyl carbocation intermediate. Subsequently, the carbocation undergoes deprotonation, leading to the formation of 1-hexene.

The novel modified FER-type zeolite catalysts (Examples 1 and 2) that are mesoporous exhibited superior performance for 1-hexanol dehydration, as compared to unmodified, commercial FER type zeolite catalysts (CP914C and MSZ35312). As shown in Table 3, under identical reaction conditions and catalyst loading weight, the site time yield was 3.11 kg_{reactant}kg⁻¹_{catalyst}hr⁻¹ for the Example 1 catalyst, which represents ~45% (i.e., 44.7%) higher than that of corresponding unmodified, commercial zeolite (2.15 kg_{reactant}kg⁻¹_{catalyst}hr⁻¹ for MSZ35312), and the site time yield was 3.09 kg_{reactant}kg⁻¹_{catalyst}hr⁻¹ for the Example 2 catalyst, which represents ~37% (i.e., 36.7%) higher than that of corresponding unmodified, commercial zeolite (2.26 kg_{reactant}kg⁻¹_{catalyst}hr⁻¹ for CP914C). The significant improvement in the site time yield brought out by the novel modified FER-type zeolite catalysts, as compared to the unmodified, commercial FER type zeolite catalysts, indicates that at mild reaction conditions (e.g., 220° C.), the property-enhancement treatment described herein was able to produce a more active catalyst.

Additionally, besides the improved catalytic activity, the novel modified FER-type zeolite catalysts (Examples 1 and 2) that are mesoporous also exhibited a higher selectivity (e.g., ~97-98%) to linear olefin isomers (e.g., 1-hexene, trans-2-hexene, cis-2-hexene, cis-3-hexene, trans-3-hexene) via isomerization, thereby suppressing the formation of byproducts such as branched olefin isomers (e.g., 4-methyl-1-pentene), and ethers (e.g., di-n-hexyl ether).

Without being bound by theory, this improved selectivity toward linear olefins may have been achieved by enhancing the porosity properties (i.e., creating and enhancing the mesoporosity) and adjusting the acid functions. The presence of mesoporous structure and the significant creation of mesopore volumes in the novel modified FER-type zeolite catalysts (Examples 1 and 2) have facilitated better diffusion of reactant and desirable products (i.e., linear olefins) within the zeolite catalyst, minimizing the likelihood of secondary reactions, such as an ether formation that can occur when reactants or products are trapped within the pores of microporous catalysts. Additionally, the presence of mesoporosity and the significant creation of mesopore volumes also have provided acid sites that are both higher in number and in strength, which improved the selective dehydration toward olefins (particularly linear olefines).

In sum, compared to the commercial FER-type dehydration catalysts (such as CP914C and MSZ35312), the novel modified FER-type zeolite catalysts (e.g., Examples 1 and 2) were more catalytically active and were more selective toward linear olefins. The superior performance of the novel modified FER-type zeolite catalysts can result in a significant improvement on the energy and cost efficiency for the catalytic reactions employing these catalysts.

### 2-Propanol Dehydration Catalytic Test:

2-propanol reaction tests were conducted in a fixed bed reactor, reactor tube with 8.48 mm internal diameter, 12.7 mm external diameter, and 210 mm length. When loading the reactor, silicon carbide was used as an inert material to position the catalytic bed at the tip of the thermocouple inside the reactor. Quartz wool was added between the inert and the catalyst to prevent displacement of the catalytic bed. When closing the reactor, a sealing washer was added. After assembly, the catalyst was pre-treated under N₂ gas flow, 100 mL/min at 470° C. for approximately 1 hour, under atmosphere pressure. Then, the liquid 2-propanol feed was controlled by a peristaltic pump. During the reaction, the preheater temperature was 250° C. and the transfer line temperature after the reactor was 250° C. The products were analyzed by gas chromatograph with flame ionization detector (FID) and thermal conductivity detector (TCD). For the screening of zeolite-based catalysts, the space velocity (WHSV) 234 h-1 and the temperature is 300° C. The diffusion limitations were eliminated in previous study. In these studies, we used reagents high purity reagent and the feed are composed of pure 2-propanol.

FIG. 4 shows a conversion of 2-propanol over various catalysts. The reaction was conducted at 300° C. with a weight hourly space velocity (WHSV) of 234 h⁻¹ using 100 mg of catalyst and a 2-propanol feed rate of 0.5 mL min-1. Reported conversion and selectivity values represent time-averaged results collected over 0-13 h on stream.

2-Propanol dehydration is an acid-catalyzed reaction that primarily produces propylene and water through unimolecular dehydration. Propylene is a key petrochemical intermediate and an important molecule for the industry. Under certain conditions, a competing bimolecular pathway leads to the formation of di-isopropyl ether as a by-product.

Braskem's proprietary catalysts-mesoporous FER-type zeolites (e.g., Example-1 and Example-2)—exhibit superior performance for 1-propanol dehydration compared to commercial FER type catalysts, CP914C from Zeolyst and MSZ35312 from ACS Material, as shown in FIG. 4 and Table 4. Under same reaction conditions, the average 2-propanol conversion is 61.82% for Example-1 and 35.24% for Example-2, which is around 25%-27% higher than that on commercial zeolites (49.48% and 27.68%). The higher conversion results from the presence of mesoporosity, which enhances the mass transport of reactants and the products and creates more accessible sites on the surface.

The analysis of the selectivity results in Table 4 highlights a significant industrial gain in the production of olefins, particularly propylene obtained from the dehydration of propanol, which shows selectivity above 96% across all evaluated catalysts. This performance demonstrates the efficiency of catalytic processes in minimizing the formation of undesired by-products such as diethyl ether and di-isopropyl ether, which appear only in very small proportions. From an industrial perspective, this high selectivity toward propylene ensures greater product purity, reduces the need for additional separation and purification steps, and optimizes operational and energy costs. In all, catalysts such as Example-1 and Example-2 which combine high selectivity with minimal by-product formation, represent a strategic advantage for petrochemical processes focused on olefin production, consolidating improvements in efficiency, sustainability, and competitiveness.

TABLE 4
Dehydration of 2-propanol over various catalysts

| | Example-1 | Example-2 | MSZ35312 | |
|---|---|---|---|---|
| | (working | (working | (ACS | CP914C |
| | example) | example) | Material) | (Zeolyst) |
| Average | 61.82 | 35.24 | 49.48 | 27.68 |
| conversion (%) | | | | |
| Selectivity (mol %) | | | | |
| Propylene | 98.62 | 97.24 | 96.58 | 98.34 |
| Di-isopropyl | 0.66 | 1.43 | 3.07 | 0.53 |
| ether | | | | |

In summary, compared to the commercial FER-type dehydration catalysts (CP914C and MSZ35312), proprietary Example-1 and Example-2 catalysts are more active and selective to the formation of linear olefins. The superior performance may bring in significant improvement on energy and cost of fatty alcohol dehydration process.

1. A modified ferrierite (FER)-type zeolite, comprising:
a FER-type zeolite, modified by a property-enhancement treatment, wherein the modified FER-type zeolite has micropores and mesopores, wherein the total mesopore volume is greater than about 0.05 cm³/g, measured by Ar adsorption isotherms obtained at 87K, or by N₂ adsorption isotherms obtained at 77K, on a Surface Area and Porosity Analyzer.

2. The modified FER-type zeolite of claim 1, wherein the modified FER-type zeolite has a total mesopore volume increase of at least about 50%, compared to the same FER-type zeolite, unmodified.

3. The modified FER-type zeolite of claim 1, wherein the modified FER-type zeolite has a BET surface area of greater than about 290 m²/g.

4. The modified FER-type zeolite of claim 1, wherein the modified FER-type zeolite has a BET surface area increase of at least about 20%, compared to the same FER-type zeolite, unmodified.

5. The modified FER-type zeolite of claim 1, wherein the FER-type zeolite comprises silicon oxide (SiO₂) and aluminum (Al₂O₃), and the SiO₂:Al₂O₃ molar ratio in the zeolite is no greater than 50.

6. The modified FER-type zeolite of claim 1, wherein the property-enhancement treatment comprises:
mixing the FER-type zeolite with an aqueous source of hydroxide ions (OH) and a surfactant to create mesopores in the zeolite; and
heating the mixture at a temperature ranging from about 50° C. to about 120° C.

7. The modified FER-type zeolite of claim 6, wherein the aqueous source of OH is NaOH, optionally wherein the concentration of NaOH ranges from 0.1 to 3M, and the amount of NaOH solution (by volume) relative to the amount of the FER-type zeolite (by weight) ranges from 10 to 100.

8. The modified FER-type zeolite of claim 6, wherein the surfactant is an ionic surfactant, optionally wherein the weight ratio between the amount of the surfactant and the amount of the FER-type zeolite ranges from 0.2 to 1.

9. The modified FER-type zeolite of claim 1, wherein the property-enhancement treatment further comprises:
calcining the modified FER-type zeolite at a temperature of about 400° C. or above for a time sufficient to remove at least part of the surfactant from the zeolite; and/or
converting the modified FER-type zeolite to a protonated form of the zeolite.

10. The modified FER-type zeolite of claim 9, wherein the converting step further comprises:
subjecting the modified FER-type zeolite to an aqueous source of ammonium ions to replace at least some metal ions in the zeolite with ammonium ions, to form an ammonium form of the zeolite; and
heating and/or calcinating the ammonium form of the zeolite at a temperature of about 300° C. or above to produce the protonated form of the zeolite.

11. A process for a fatty alcohol dehydration, comprising:
providing a feedstock containing a fatty alcohol; and
dehydrating the fatty alcohol in the feedstock in the presence of a modified ferrierite (FER)-type zeolite catalyst to obtain a dehydration product,
wherein the modified FER-type zeolite comprises:
a FER-type zeolite, modified by a property-enhancement treatment, wherein the modified FER-type zeolite has micropores and mesopores, wherein the total mesopore volume is greater than about 0.05 cm³/g, measured by Ar adsorption isotherms obtained at 87K, or by N₂ adsorption isotherms obtained at 77K, on a Surface Area and Porosity Analyzer.

12-13. (canceled)

14. The process of claim 11, wherein the fatty alcohol comprises one or more OH groups attached to an aliphatic chain of C₄-C₂₆ carbon atoms, or
wherein the fatty alcohol comprises one or more OH groups attached to an aliphatic chain of C₂-C₂₆ carbon atoms.

15. The process of claim 14, wherein the fatty alcohol is a C₄-C₈ alcohol.

16-25. (canceled)

26. The process of claim 11, wherein the fatty alcohol is 1-hexanol, and the primary dehydration product is a C₆ linear olefin or its isomer.

27-36. (canceled)

37. A fatty alcohol dehydration product prepared according to the process of claim 11, wherein the dehydration product contains:
at least 85 mol % of a linear olefin or its isomer,
no more than about 5 mol % branched olefin, and/or
no more than about 5 mol % ether.

38. (canceled)

39. The modified FER-type zeolite of claim 8, wherein the ionic surfactant is a cationic surfactant.

40. The process of claim 14, wherein the fatty alcohol comprises one or more OH groups attached to an aliphatic chain of C₃-C₂₆ carbon atoms.
